(19) United States
(12) Patent Application Publication
(10) Pub. No.: US 2017/0348541 A1
(43) Pub. Date: Dec. 7, 2017

(54) Portable Light Therapy Assembly

(71) Applicant: Jared Kenneth Post, Zumbrota, MN (US)

(72) Inventor: Jared Kenneth Post, Zumbrota, MN (US)

(21) Appl. No.: 15/609,844

(22) Filed: May 31, 2017

Related U.S. Application Data
(60) Provisional application No. 62/344,196, filed on Jun. 1, 2016.

(51) Int. Cl. A61N 5/06; A61N 5/067

(52) U.S. Cl. CPC A61N 5/06; A61N 2005/067; A61N 2005/0626; A61N 2005/0663

(57) ABSTRACT

A portable light therapy treatment assembly includes a housing and a control unit that is mounted in the housing. A power supply is mounted in the housing and electrically coupled to the control unit. A light emitter is mounted in the housing and emits light outwardly of the first end when the light emitter is turned on. The light emitter is electrically coupled to the control unit. The control unit is programmed to allow the light emitter to be used a pre-selected number of usages. Each of the usages defines a treatment and the total number of usages defines a prescription. The control unit requires a reset input when the prescription has been fully utilized.

### CROSS-REFERENCE TO RELATED APPLICATIONS

This application takes benefit under 35 U.S.C. Section 119(e) of Unites States Provisional Application 62/344,196 filed on Jun. 1, 2016 and incorporates such fully herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to light therapy treatment devices and more particularly pertains to a new light therapy treatment device for allowing a patient requiring therapeutic light treatments to perform such treatments on themselves and at their own convenience. Light therapy has several uses and one such is use is within the chiropractic and physical therapy industries to treat pain and promote healing of soft tissues. Because light therapy devices have been very large and expensive, a patient has been required to visit a caregiver's treatment office. This can be time consuming and inconvenient because several visits in a single week over a period of multiple weeks are typically required to maximize the benefits from the light therapy. Thus, patients often do not receive the maximum number of light therapy treatments from which they would benefit.

### BRIEF SUMMARY OF THE INVENTION

An embodiment of the disclosure meets the needs presented above by generally comprising a housing that has a first end, a second end and a perimeter wall extending between the first and second ends. A control unit is mounted in the housing and a power supply is mounted in the housing and electrically coupled to the control unit. A light emitter is mounted in the housing and emits light outwardly of the first end when the light emitter is turned on. The light emitter is electrically coupled to the control unit. The control unit is programmed to allow the light emitter to be used a pre-selected number of usages. Each of the usages defines a treatment and the total number of usages defines a prescription. The control unit requires a reset input when the prescription has been fully utilized.

In another embodiment of the disclosure includes a method of providing a housing a housing having a first end, a second end and a perimeter wall extending between the first and second ends. A control unit is mounted in the housing and a power supply mounted in the housing is electrically coupled to the control unit. The power supply comprises a rechargeable battery. A light emitter is mounted in the housing and emits light outwardly of the first end when the light emitter is turned on. The light emitter is electrically coupled to the control unit. A power switch is mounted on the housing and is operationally coupled to the control unit. The power switch is actuated to allow the control unit to turn the light emitters on or off. The control unit is programmed to allow the light emitter to be used a pre-selected number of usages wherein each of the usages defines a treatment and the total number of usages defining a prescription. The power switch is actuated to initiate one of the treatments. The steps of actuating the power switch and initiating one of the treatments is repeated until the prescription has been fully utilized. The control unit must then be reset with a new prescription to allow the light emitter to be used again.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a portable light therapy treatment assembly according to an embodiment of the disclosure.

FIG. 2 is a top view of an embodiment of the disclosure.

FIG. 3 is a back view of an embodiment of the disclosure.

FIG. 4 is a cross-sectional view of an embodiment of the disclosure taken along line 4-4 of FIG. 3.

FIG. 5 is a schematic view of an embodiment of the disclosure.

FIG. 6 is a schematic view of an embodiment of the disclosure.

### DETAILED DESCRIPTION OF THE INVENTION

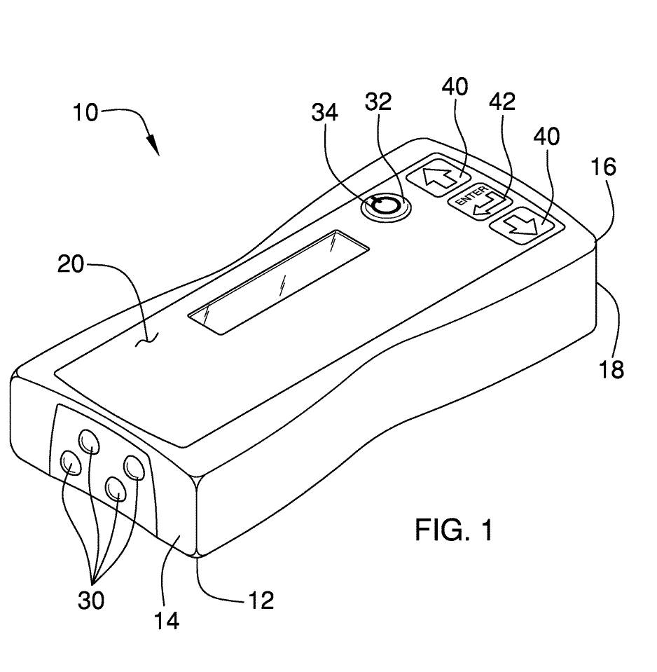

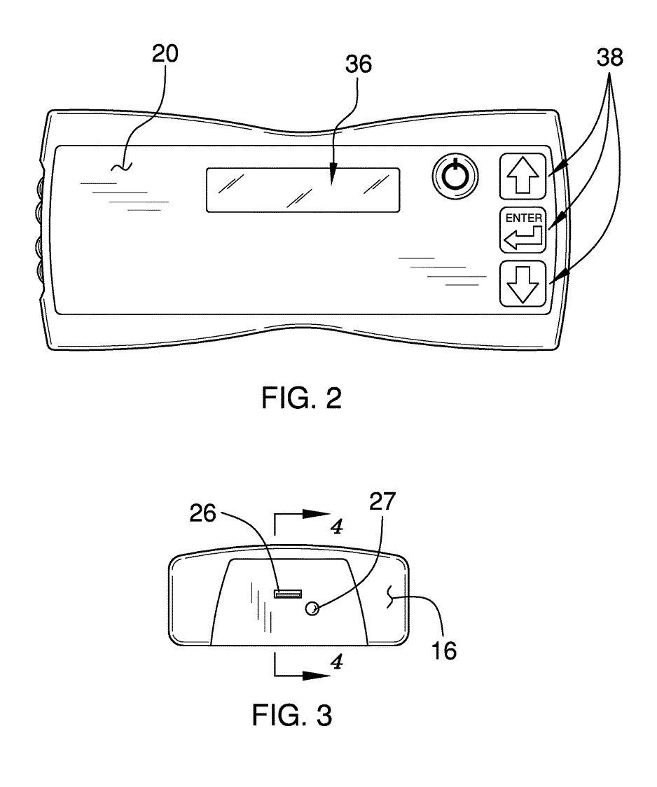

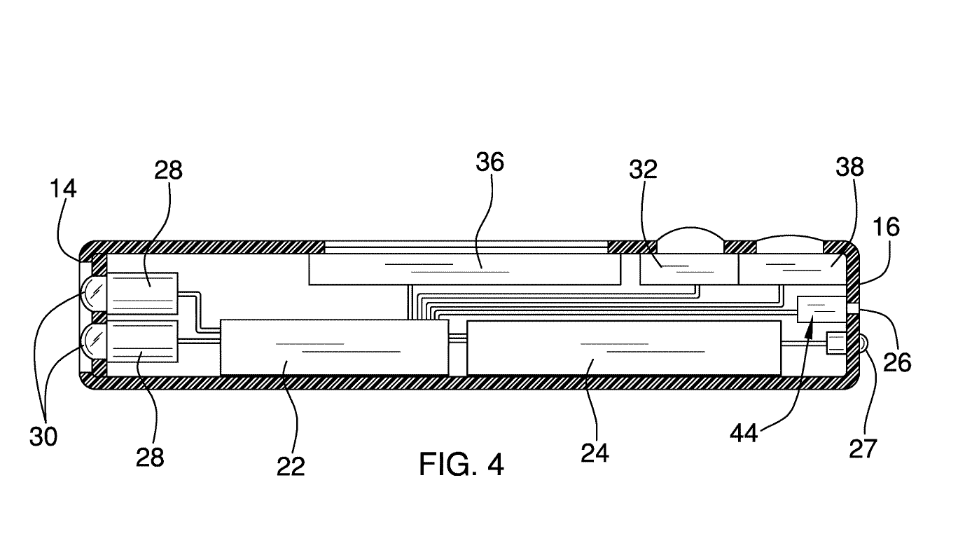

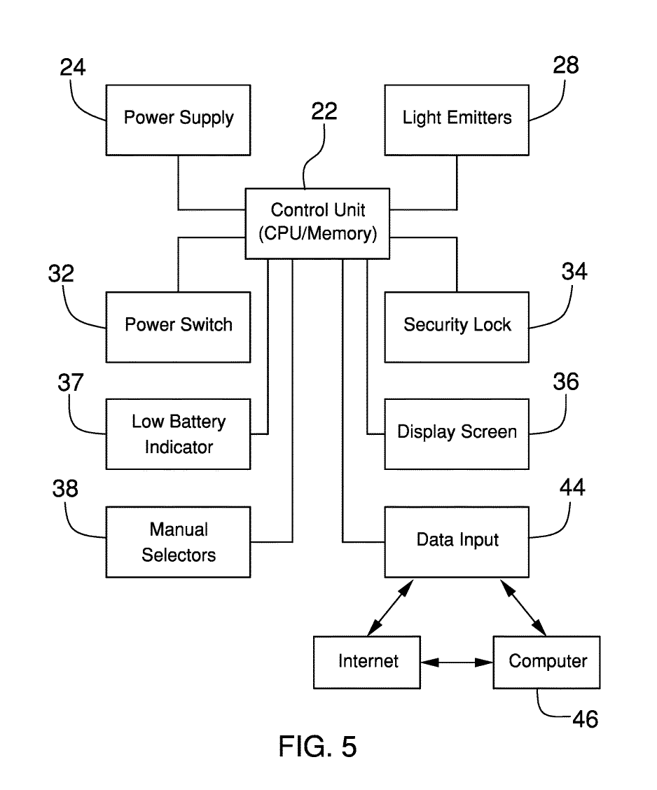

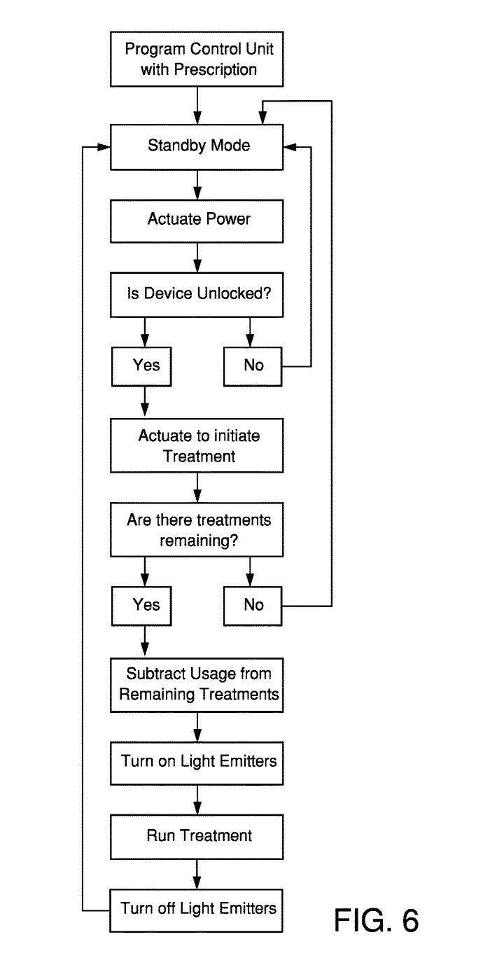

With reference now to the drawings, and in particular to FIGS. 1 through 6 thereof, a new light therapy treatment device embodying the principles and concepts of an embodiment of the disclosure and generally designated by the reference numeral 10 will be described.

As best illustrated in FIGS. 1 through 6, the portable light therapy treatment assembly 10 generally comprises a housing 12 that has a first end 14, a second end 16 and a perimeter wall 18 extending between the first 14 and second 16 ends. The perimeter wall 12 includes an upper wall 20 and the housing 12 comprises an overall volume of less than about 150 in³, preferably less than 100 in³, and more preferably less than 50 in³ so that the housing 12 is easily portable and can be easily manipulated by a person using only one hand.

A control unit 22 is mounted in the housing. The control unit 22 generally will include a processor (CPU) and a memory module. The memory module will specifically include at least some non-volatile memory so that information may be stored after the powering down of the assembly 10. A power supply 24 is mounted in the housing 12 and is electrically coupled to the control unit 22. More particularly, the power supply 24 may comprise one or more rechargeable batteries. A power port 26 is electrically coupled to the power supply, or rechargeable battery. The power port 26 extends through the housing 12 and may, additionally, comprise a data port such as a USB port. The control unit 22 may also be GPS enabled to allow the assembly 10 to be located remotely should it be lost or not be returned to a provider of the assembly 10. A power indicator light 27 may be provided which is mounted on the housing 12 is electrically coupled to the control unit 22 to indicate when power supply is running low, such as around 10% or less of total battery capacity.

A light emitter 28 is mounted in the housing 12 and emits light outwardly of the housing, and more particularly may emit the light from the first end 14, when the light emitter 28 is turned on. The housing 12 may include a pivoting head in which the light emitter 28 is mounted to allow the directly of the emitted light to be selectively altered by up to 90°. The light emitter 28 is electrically coupled to the control unit 22. The light emitter 28 typically will comprise a 635 mm laser (red emitted light), though other types of lasers or light sources may be utilized as well as other light wavelengths wherein the light emitter 28 is calibrated to emit light within a specific color range dependent upon the type of therapy being performed. A lens 30 is mounted on the housing 12 through which light from the light emitter 28 passes. The lens 30 focuses and intensifies the emitted light and will typically comprise a collimating lens and may further include a Powell lens used for generating lines of light. As can be seen in the Figures, the light emitter 28 and lens 30 may include a plurality of light emitters 30 and lenses 32. The light emitter(s) 30 is adjustable to emit light at a selected frequency (pulse rate of light emission) according to the type of therapy to be effected. The control unit 22 is programmable to adjust this frequency as needed which may vary from 0 Hz to 1 GHz. During a prescribed treatment, the frequency itself may be altered so that multiple frequencies are used during a single treatment. The light emitters 30 may also be provided with intensity adjustments to alter the lux levels of the light emitters 30.

A power switch 32 is mounted on the housing 12 and is operationally coupled to the control unit 22. The power switch 32 is actuated to allow the control unit 32 to turn on or off the light emitters 28. The power switch 32 includes a security lock 34 to inhibit unauthorized usage of the light emitter 28. As can be seen in FIG. 1, the security lock 34 may comprise a mechanical key lock which requires a key to be inserted into the security lock and rotated to turn on the control unit and/or allow the control unit 22 to turn on the light emitters. Thus, it should be understood that the security lock 34 may be used for either allowing the powering of the control unit 22 or allowing the control unit 22 to turn on the light emitters 28. Some security locks, as understood below, will generally require the control unit 22 to be turned on before the security lock 34 is actuated so that a security input can be analyzed for authenticity. For example, alternative security locks might be utilized such as entering a numerical or alphanumerical code; biometric security inputs such as fingerprint scanners or voice recognition software; wireless activations such as from cellular phones, key fobs and RFID chips; and other conventional security means. The security lock 34 ensures that unwanted usage, particularly by children, does not occur.

The control unit 22 is programmed to allow the light emitter 28 to be used a pre-selected number of usages. Each of the usages defines a treatment with the total number of usages defining a prescription. After the prescription has been fully utilized, that is that all treatments have been used, the control unit 22 will require a reset input to create a new prescription which may be used. In this manner, a prescribing caretaker can ensure that the user of the assembly 10 only uses the light emitters 28 a pre-determined number of times. When the prescription is entered into the control unit, it will include instructions for the control unit 22 with respect to the light frequency, duration of a treatment (usage) and number of treatments. Additionally, the control unit 22 may be programmed with a prescription that only allows usage during a specific time frame. For example, should a person be given six treatments to use over a specific time frame but does not use all six within that time frame, the assembly 10 will not allow usage of the unused treatments.

A display screen 36 is mounted on the housing 12 and is electrically coupled to the control unit 22. The display 36 may be mounted on or visible through the upper wall 20. The display screen 36 is used for communicating with and displaying information from the control unit 22. Foremost, the display screen 36 will depict a remaining number of the treatments remaining. However, the display screen 36 may be used for displaying other useful information. In particular, the display screen 36 will display the prescription as it is being entered into the control unit 22 or as is will be performed/executed by the assembly 10 for the user thereof. Since a light therapy treatment is done over the course of a predetermined amount of time, typically between 1.0 minutes and 30 minutes, the display screen 36 may be used to indicate the time remaining in a treatment. If the control unit 22 includes a programming system of options for treatments, these options, such in the form of folders and subfolders (sub-options), will be visible on the display screen 36. This would allow a caregiver to program the number of treatments, the frequency of the emitted light, and duration of the treatment, for example. Additionally, the display screen 36 may be used for instructing a user of the assembly 10 to perform additionally prescribed routines such as performing a period of exercise in addition to the light therapy.

The display screen 36 may also be used for inputting an alphanumeric code should the assembly 10 require such to overcome the security lock. Furthermore, the display screen 36 may comprise a touch screen to function as an input for control unit. If the display screen 36 is not a touch screen, the assembly 10 will typically include a manual selector 38 that is mounted on the housing 12 and is operationally coupled to the control unit 22. The manual selector 38 is configured to start, finish and access the treatments and may also be used for inputting such into the control unit 22. As can be seen in the Figures, a plurality of manual selectors may be provided including, for example, scrolling selectors 40 and an enter actuator 42.

A data input 44 is mounted in the housing and is electrically coupled to the control unit to allow the control unit 22 to be programmed and to retrieve stored data from the control unit. The data input 44 may comprise a USB port 26, as described above and also used for recharging the power supply 24. The data input 44 may alternatively or additionally comprise a transceiver utilizing wireless frequencies such as Cellular Signals, Bluetooth, Wi-Fi, or near-field communication protocols. Wireless communication may further allow remote programming and access to the control unit 22 via the Internet. Regardless of the type of data input 44 utilized, the data input 44 will allow communication between the control unit 22 and a computer 46. This will allow the computer to program the control unit 22 with a particular treatment regimen and also to upload, from the stored memory of the control unit 22, data related to if and when the treatments were performed. The computer 46 may then also store overall treatment information related to a patient such as the number, type and efficacy of the treatments received by a specific patient. Further information may indicate when the treatments were performed to determine if the patient is properly chronologically spacing them from each other. It should be understood that the control unit 22 may be programmed allow treatments to take place only after a sufficient amount of time has elapsed between treatments.

In use, a caregiver such as a physician, chiropractor or other authorized person will program the control unit to allow the light emitter 28 to be used a pre-selected number of treatments and will further program the control unit 22 as to what the treatment will consist of. The caregiver may have a special access code or program recognized by the control unit 22 to allow prescription access. The caregiver then provides the assembly 10 to the patient (user) so that the patient may take the assembly 10 with them to use at their convenience and in accordance with the prescription. The patient will initiate the treatments, on a schedule provided by the caregiver, until such time as all of the treatments have been fully utilized. “Fully utilized” herein may include, for example, a minimal amount of time of treatment or as little as simply starting a treatment. In this manner a patient would not be allowed to turn off the light emitter 28 before the end of the treatment and thereafter restart the treatment from the beginning. Alternatively each treatment may be considered a fully allotted amount of time. As an example, the patient may only use 10 minutes of a 15 minute prescribed treatment and will be able to later use the remaining 5 minutes of that treatment. Once all of the treatments have been utilized, the caregiver may provide another prescription or the assembly 10 may be returned to the caregiver. Utilizing the internet and depending on either a wired or wireless connection, the control unit 22 may communicate with a computer 46 accessed by the caregiver to monitor usage of the assembly 10, either constantly or intermittently as data is uploaded from the control unit 22 to the computer 46. Using a remote connection would allow the caregiver to remotely reinitiate a prescription or prevent further usage of the assembly 10.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A portable light therapy treatment providing assembly comprising:
a housing;
a control unit being mounted in said housing, a power supply being mounted in said housing and being electrically coupled to said control unit;
a light emitter being mounted in said housing and emitting light outwardly of said housing when said light emitter is turned on, said light emitter being electrically coupled to said control unit;
said control unit being programmed to allow said light emitter to be used a pre-selected number of usages, each of said usages defining a treatment and said total number of usages defining a prescription, said control unit requiring a reset input when said prescription has been fully utilized.

2. The portable light therapy treatment providing assembly according to claim 1, wherein said power supply comprise a rechargeable battery.

3. The portable light therapy treatment providing assembly according to claim 2, further including a power port being electrically coupled to said rechargeable battery.

4. The portable light therapy treatment providing assembly according to claim 1, further including a lens being mounted on said housing through which light from said light emitter passes through.

5. The portable light therapy treatment providing assembly according to claim 1, wherein said light emitter is adjustable to emit light at a selected frequency, said control unit being programmable to selectively choose a frequency of the light emitted from said light emitter for each said treatment.

6. The portable light therapy treatment providing assembly according to claim 1, further including a power switch being mounted on said housing and being operationally coupled to said control unit, said power switch being actuated to allow said control unit to turn said light emitter on or off.

7. The portable light therapy treatment providing assembly according to claim 6, wherein said power switch includes a security lock to inhibit unauthorized usage of said light emitter.

8. The portable light therapy treatment providing assembly according to claim 1, further including a display screen being mounted on said housing and being electrically coupled to said control unit, said display screen displaying a remaining number of said treatments remaining.

9. The portable light therapy treatment providing assembly according to claim 8, wherein said display screen displays said prescription.

10. The portable light therapy treatment providing assembly according to claim 1, further including a manual selector being mounted on said housing and being operationally coupled to said control unit, said manual selector being configured to start, finish and access said treatments.

11. The portable light therapy treatment providing assembly according to claim 1, further including a data input being mounted in said housing and being electrically coupled to said control unit to allow said control unit to be programmed and to retrieve stored data from said control unit.

12. A portable light therapy treatment providing assembly comprising:
a housing having a first end, a second end and a perimeter wall extending between said first and second ends, said perimeter wall including an upper wall, said housing having an overall volume of less than 100 cubic inches;
a control unit being mounted in said housing, a power supply being mounted in said housing and being electrically coupled to said control unit, said power supply comprising a rechargeable battery, a power port being electrically coupled to said rechargeable battery;
a light emitter being mounted in said housing and emitting light outwardly of said first end when said light emitter is turned on, said light emitter being electrically coupled to said control unit;
a lens being mounted on said housing through which light from said light emitter passes through;
said control unit being programmed to allow said light emitter to be used a pre-selected number of usages, each of said usages defining a treatment and said total number of usages defining a prescription, said control unit requiring a reset input when said prescription has been fully utilized;
said light emitter being adjustable to emit light at a selected frequency, said control unit being programmable to selectively choose a frequency of the light emitted from said light emitter for each said treatment;
a power switch being mounted on said housing and being operationally coupled to said control unit, said power switch being actuated to all allow said control unit to turn said light emitter on or off, said power switch including a security lock to inhibit unauthorized usage of said light emitter;
a display screen being mounted on said housing and being electrically coupled to said control unit, said display screen displaying a remaining number of said treatments remaining, said display screen displaying said prescription;
a manual selector being mounted on said housing and being operationally coupled to said control unit, said manual selector being configured to start, finish and access said treatments; and
a data input being mounted in said housing and being electrically coupled to said control unit to allow said control unit to be programmed and to retrieve stored data from said control unit.

13. A method of portably administering light therapy treatments comprising the steps of:
providing a housing and a control unit being mounted in said housing, a power supply being mounted in said housing and being electrically coupled to said control unit, said power supply comprising a rechargeable battery, a light emitter being mounted in said housing and emitting light outwardly of said housing when said light emitter is turned on, said light emitter being electrically coupled to said control unit, a power switch being mounted on said housing and being operationally coupled to said control unit, said power switch being actuated to allow said control unit to turn said light emitters on or off;
programming said control unit to allow said light emitter to be used a pre-selected number of usages, each of said usages defining a treatment and said total number of usages defining a prescription;
actuating said power switch;
initiating one of said treatments;
repeating the steps of actuating said power switch and initiating one of said treatments until said prescription has been fully utilized; and
resetting said control unit with a new prescription.

14. The method according to claim 13, wherein the step of providing said housing further includes the step of providing said light emitter being adjustable to emit light at a selected frequency, said control unit being programmable to selectively choose a frequency of the light emitted from said light emitter for each said treatment.

15. The method according to claim 13, further including:
wherein the step of providing said housing further includes the step of said power switch including a security lock to inhibit unauthorized usage of said light emitter;
unlocking said security lock to allow said control unit to turn on said light emitter.

16. The method according to claim 13, wherein the step of providing said housing further includes the step of providing a display screen being mounted on said housing and being electrically coupled to said control unit, said display screen displaying a remaining number of said treatments remaining, said display screen displaying said prescription.

17. The method according to claim 13, wherein the step of providing said housing further includes the step of providing a manual selector being mounted on said housing and being operationally coupled to said control unit, said manual selector being configured to start, finish and access said treatments.

18. The method according to claim 13, wherein the step of providing said housing further includes the step of providing a data input being mounted in said housing and being electrically coupled to said control unit to allow said control unit to be programmed and to retrieve stored data from said control unit.
